(19) United States
(12) Patent Application Publication
(10) Pub. No.: US 2021/0386966 A1
(43) Pub. Date: Dec. 16, 2021

(54) METHODS FOR IN SITU FABRICATION OF SENSOR ELECTRODES, AND MEDICAL SYSTEMS AND DEVICES EMPLOYING SUCH SENSOR ELECTRODES

(71) Applicant: UNIVERSITY OF MARYLAND, COLLEGE PARK, College Park, MD (US)

(72) Inventors: Ryan HUISZOON, Silver Spring, MD (US); Sangwook CHU, San Jose, CA (US); Reza GHODSSI, Potomac, MD (US)

(21) Appl. No.: 17/347,994

(22) Filed: Jun. 15, 2021

Related U.S. Application Data
(60) Provisional application No. 63/039,367, filed on Jun. 15, 2020.

(51) Int. Cl. A61M 25/00

(52) U.S. Cl. CPC A61M 25/0012; A61M 25/0017; A61M 2210/1078; A61M 2025/0048; A61M 25/0045

(57) ABSTRACT

Sensor electrodes are fabricated in situ within or on a surface of a medical device. For example, a catheter can have a lumen extending between first and second longitudinal ends of the catheter. A patterning mold can be inserted into the lumen via the first longitudinal end of the catheter such that first and second surface portions of the lumen are exposed from the patterning mold and remaining surface portions of the lumen are covered by and in contact with the patterning mold. A first electrode layer can be formed on the first and second surface portions exposed from the patterning mold using electroless deposition. After the forming, the patterning mold can be removed from the lumen. Additional electrode layers can be formed on the first electrode layer, for example, via electroplating. In some embodiments, the electrode layers can be used for detection of bacterial biofilm growth.

### CROSS-REFERENCE TO RELATED APPLICATION

The present application claims the benefit of U.S. Provisional Application No. 63/039,367, filed Jun. 15, 2020, entitled “System, Device, and Method for Detecting and Mitigating Biofilms and Methods of Making the Same,” which is incorporated by reference herein in its entirety.

### STATEMENT REGARDING FEDERALLY SPONSORED RESEARCH

This invention was made with government support under Award No. ECCS1809436 awarded by the National Science Foundation (NSF). The government has certain rights in the invention.

### FIELD

The present disclosure relates generally to medical systems and devices, and more particularly, to methods for in situ fabrication of sensor electrodes and medical systems and devices with such sensor electrodes.

### BACKGROUND

Biofilms form when bacteria adhere to a hydrated surface and, at a threshold population, encase themselves in a protective extracellular matrix (ECM), which increases their resistance to stressors such as antimicrobial treatments. Bacteria in biofilms require 500-5000× higher doses of antibiotics for removal compared to their planktonic counterparts. Bacterial cells may slough off of mature biofilms and spread throughout the environment. Thus, biofilms can serve as a source of recurring infections in human healthcare, particularly when they form on implanted or inserted medical devices. For example, urinary catheters, which can be colonized by bacterial biofilms leading to catheter-associated urinary tract infections (CAUTIs), exhibit an infection rate of 5-7% per day of implantation. Guidelines provided by the Centers for Disease Control and Prevention (CDC) for the prevention of CAUTI suggest that catheters should be replaced based on clinical indications. However, symptoms indicative of CAUTI may not be apparent until an infection has reached a certain degree of severity, which, in turn, may require more aggressive treatment modalities (e.g., a higher dose of antibiotics). Providing medical devices with sensors for early detection of biofilm formation and proliferation can help in avoiding severe infections and the concomitant aggressive treatment options.

### SUMMARY

Embodiments of the disclosed subject matter provide methods for in situ fabrication of sensor electrodes, and medical systems and devices employing such sensor electrodes. In prior fabrication techniques, thin-film metal electrodes are formed on a flexible planar substrate, which is subsequently deformed (e.g., bent or rolled) for coupling to a non-planar (e.g., curved) surface of a medical device, such as the internal lumen of a urinary catheter. However, the deformation of the planar substrate can cause fracture or cracking of the metal electrodes. Moreover, the ability of the planar substrate to conform to a complex surface (e.g., having a non-arcuate, non-planar shape) may be limited. The disclosed methods allow for in situ fabrication of the sensor electrodes on the surface of the medical device itself, for example, on an internal surface of thereof. In contrast, conventional fabrication techniques, such as photolithography and physical vapor deposition (PVD), may require external access to the surface and thus be incapable of forming electrodes within a medical device.

In embodiments, a patterning mold selectively screens a surface of the medical device for direct plating of one or more metal layers of electrodes thereon (or thereover). For example, the patterning mold can be inserted into a lumen, recess, or other opening of the medical device so as to contact parts of an internal surface thereof while leaving other parts of the internal surface exposed. An aqueous electroless plating process can then be performed to deposit one or more metal layer directly on (or over) the internal surface while the patterning mold remains in place. In some embodiments, additional metal layers can be formed upon (or over) the electroless deposited metal layer(s), for example, by performing a metal immersion process, by another electroless plating process, or by electroplating. In certain exemplary embodiments, the medical device can be catheter (e.g., urinary catheter), and the electrodes can be formed over at least an internal wall of a lumen (e.g., drainage lumen) of the catheter. However, in some embodiments, the medical device can be any other type of indwelling medical device.

In one or more embodiments, a method of fabricating a catheter with in situ sensor electrodes can comprise inserting a patterning mold into an internal lumen of the catheter. The internal lumen can extend between first and second longitudinal ends of the catheter. The patterning mold can be inserted via the first longitudinal end of the catheter. First and second surface portions of the internal lumen are exposed from the patterning mold while remaining surface portions of the internal lumen are covered by and in contact with the patterning mold. The method can further comprise forming a first electrode layer over the first and second surface portions exposed from the patterning mold using electroless deposition. The first electrode layer can comprise a first metal. The method can also comprise, after the forming, removing the patterning mold from the internal lumen.

In one or more embodiments, a system can comprise a catheter, a first electrode, and a second electrode. The catheter can be constructed to be disposed within an in vivo environment. The catheter can have a first longitudinal end, a second longitudinal, and an internal lumen extending between the first and second longitudinal ends. The first and second electrodes can be integrally formed over respective surface portions of the internal lumen. Each of the first and second electrodes can comprise an electroless-deposited layer of a first metal.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

Examples will hereinafter be described with reference to the accompanying drawings, which have not necessarily been drawn to scale. Where applicable, some elements may be simplified or otherwise not illustrated in order to assist in the illustration and description of underlying features. Throughout the figures, like reference numerals denote like elements.

FIG. 1A is a simplified illustration of an exemplary medical system with in situ fabricated electrodes for detecting bacteria biofilms, according to one or more embodiments of the disclosed subject matter.

FIG. 1B depicts a generalized example of a computing environment in which the disclosed technologies may be implemented.

FIG. 2A is a simplified view of the urinary catheter with in situ fabricated electrodes, according to one or more embodiments of the disclosed subject matter.

FIGS. 2B-2C are cross-sectional views (perpendicular to a longitudinal direction L) of the urinary catheter of FIG. 2A.

FIG. 2D is a cross-sectional view of a urinary catheter with internal and external electrodes, according to one or more embodiments of the disclosed subject matter.

FIG. 2E is a cross-sectional view of a urinary catheter having a non-circular internal lumen, according to one or more embodiments of the disclosed subject matter.

FIG. 3A is a cross-sectional view of a urinary catheter with in situ fabricated electrodes comprising multiple metal layers, according to one or more embodiments of the disclosed subject matter.

FIG. 3B is a cross-sectional view of a urinary catheter with in situ fabricated electrodes formed over an adhesion promoting layer, according to one or more embodiments of the disclosed subject matter.

FIG. 3C is a cross-sectional view of a urinary catheter with a protection layer formed over in situ fabricated electrodes, according to one or more embodiments of the disclosed subject matter.

FIG. 3D is a cross-sectional view of a urinary catheter having in situ fabricated electrodes formed of multiple metal layers, an adhesion promoting layer, and a protection layer, according to one or more embodiments of the disclosed subject matter.

FIG. 4A is a simplified view of a urinary catheter with in situ fabricated electrodes having an interdigitated electrode pattern, according to one or more embodiments of the disclosed subject matter.

FIGS. 4B-4C are cross-sectional views of the urinary catheter of FIG. 4A.

FIG. 5 is a process flow diagram for an exemplary method of in situ fabricating sensor electrodes within or on a medical device, according to one or more embodiments of the disclosed subject matter.

FIGS. 6A-6B illustrate a urinary catheter and a patterning mold in an unassembled configuration and assembled configuration, respectively, prior to in situ fabrication of electrodes, according to one or more embodiments of the disclosed subject matter.

FIGS. 6C-6D are cross-sectional views of the assembly of the urinary catheter and patterning mold of FIG. 6B.

FIG. 6E is a cross-sectional view of an assembly of a urinary catheter and another patterning mold, according to one or more embodiments of the disclosed subject matter.

FIG. 7A illustrates an assembly of a urinary catheter and a patterning mold for in situ fabrication of interdigitated electrodes, according to one or more embodiments of the disclosed subject matter.

FIGS. 7B-7C are cross-sectional views of the assembly of the urinary catheter and patterning mold of FIG. 7A.

FIGS. 8A-8B are cross-sectional views of an assembly of a urinary catheter and a patterning mold having removable sections, during electroless deposition and during removal, respectively, according to one or more embodiments of the disclosed subject matter.

FIGS. 9A-9B are cross-sectional views of an assembly of a urinary catheter and a reconfigurable patterning mold, during electroless deposition and during removal, respectively, according to one or more embodiments of the disclosed subject matter.

FIG. 10 is a simplified schematic diagram of a setup for biofilm growth experiments in a catheter device employing in situ fabricated electrodes.

FIG. 11A shows a patterning mold that was used for selective screening in an electroless plating process to in situ fabricate electrodes within the urinary catheter of FIG. 11B.

FIG. 11B shows a cross-sectional view of a urinary catheter with in situ fabricated electrodes.

FIGS. 11C-11E are scanning electron microscopy (SEM) characterizations of layer formation, with energy dispersive X-ray spectroscopy (EDS) scans for Au, Cl, and Si, from regions (i), (ii), and (iii), respectively, for the urinary catheter of FIG. 11B.

FIG. 11F is a graph comparing EDS spectra acquired from surface portions of the urinary catheter of FIG. 11B screened by the patterning mold (black dotted) and exposed by the patterning mold to electroless plating (red solid).

FIG. 12A shows impedance spectra measured by in situ fabricated electrodes at the beginning (blue) and at the end (red) of the test period (24 hours later) for a bacteria biofilm sample.

FIG. 12B shows impedance spectra measured by in situ fabricated electrodes at the beginning (blue) and at the end (red) of the test period (24 hours later) for a control sample of lysogeny broth (LB) media only.

FIG. 12C shows impedance spectra measured by in situ fabricated electrodes before and after use in detecting the bacteria biofilm sample (FIG. 12A) using LB media only.

FIG. 12D shows impedance spectra measured by in situ fabricated electrodes before and after use in detecting the control sample (FIG. 12B) using LB media only.

FIG. 13 shows changes in impedance measured by in situ fabricated electrodes (“in situ device”) and by separate electrodes formed on a flexible substrate (“flexible device”) for biofilm and control samples.

### DETAILED DESCRIPTION

### General Considerations

For purposes of this description, certain aspects, advantages, and novel features of the embodiments of this disclosure are described herein. The disclosed methods and systems should not be construed as being limiting in any way. Instead, the present disclosure is directed toward all novel and nonobvious features and aspects of the various disclosed embodiments, alone and in various combinations and sub-combinations with one another. The methods and systems are not limited to any specific aspect or feature or combination thereof, nor do the disclosed embodiments require that any one or more specific advantages be present, or problems be solved. The technologies from any embodiment or example can be combined with the technologies described in any one or more of the other embodiments or examples. In view of the many possible embodiments to which the principles of the disclosed technology may be applied, it should be recognized that the illustrated embodiments are exemplary only and should not be taken as limiting the scope of the disclosed technology.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including 10% of that value are intended, unless explicitly stated otherwise.

Directions and other relative references may be used to facilitate discussion of the drawings and principles herein, but are not intended to be limiting. For example, certain terms may be used such as “inner,” “outer,”, “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

Material layers may be described as being “on” or being “over” other material layers. As used herein, a first layer that is “on” a second layer means that the first and second layers are in direct contact with each other (e.g., along a direction parallel to a surface normal of the first layer). As used herein, a first layer that is “over” a second layer means that one or more intervening layers can be disposed between the first and second layers (e.g., along a direction parallel to a surface normal of the first layer), each of the first and second layers being in direct contact with at least one of the intervening layers (e.g., along a direction parallel to a surface normal of the first layer).

As used herein, “comprising” means “including,” and the singular forms “a” or “an” or “the” include plural references unless the context clearly dictates otherwise. The term “or” refers to a single element of stated alternative elements or a combination of two or more elements, unless the context clearly indicates otherwise.

Although there are alternatives for various components, parameters, operating conditions, etc. set forth herein, that does not mean that those alternatives are necessarily equivalent and/or perform equally well. Nor does it mean that the alternatives are listed in a preferred order, unless stated otherwise. Unless stated otherwise, any of the groups defined below can be substituted or unsubstituted.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Features of the presently disclosed subject matter will be apparent from the following detailed description and the appended claims.

### Introduction

In embodiments of the disclosed subject matter, a method for in situ fabrication of electrodes for a medical device can employ a patterning mold for selectively screening parts of the medical device surface for electroless deposition of one or more electrode layers directly thereon, thereby avoiding the manufacturing challenges associated with fabricating electrodes on a separate flexible substrate and subsequently conforming the substrate to a complex surface (e.g., non-planar) of the medical device. In some embodiments, additional electrode layers can be formed on or over the electroless deposited layers, for example, by performing a metal immersion process, by another electroless plating process, or by electroplating. In some embodiments, the electrode deposition surface can be an internal surface of the medical device that is not otherwise accessible for performance of conventional fabrication techniques, such as photolithography and physical vapor deposition (PVD). For example, the medical device can be catheter (e.g., urinary catheter), and the electrodes can be formed over at least an internal wall of a lumen (e.g., drainage lumen) of the catheter.

In some embodiments, the in situ fabricated electrodes can be used for detection of bacterial biofilm growth, for example, by sensing changes in impedance in response to an applied AC voltage signal. For example, bacterial biofilms on catheters can lead to severe catheter-associated urinary tract infections. Embodiments of the disclosed subject matter can enable biofilm monitoring via real-time impedance measurement without interfering with the operation of the catheter. For example, the disclosed in situ fabrication process can yield isolated gold electrodes directly adhered to the inner lumen of a urinary catheter. In some fabricated examples, urinary catheters with integrated sensors displayed a significant decrease in impedance of approximately 10% over the 24-hour biofilm growth period, as compared to a slight increase of 19% in control experiment, thereby confirming the viability of this approach.

### Medical System for Biofilm Detection

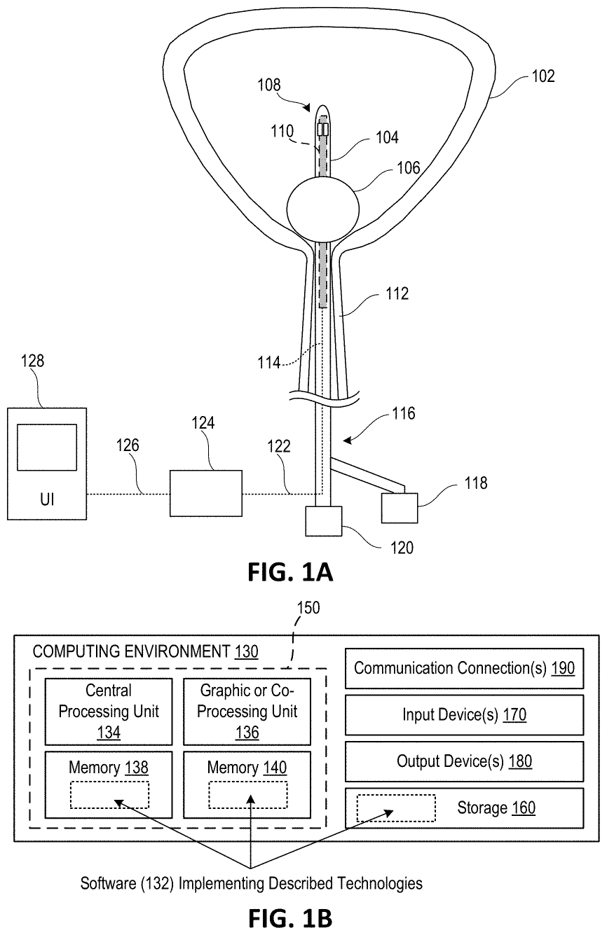

FIG. 1A illustrates an exemplary medical system with in situ fabricated electrodes for detecting bacteria biofilms in an in vivo environment, e.g., within the bladder 102 of a patient. The medical system can include a urinary catheter 104, which, in the illustrated example, is configured as a Foley catheter with a retention balloon 106 that is inflated via balloon port 118 at a first end 116 of the catheter 104. Once inflated, the retention balloon 106 sits within the patient's bladder 102 to prevent accidental removal therefrom via the urethra 112. However, other catheter configurations are also possible according to one or more embodiments. For example, the urinary catheter could be a Coude catheter, a Councill tip catheter, or a triple lumen catheter. In some embodiments, the catheter 104 can be formed of silicone rubber, latex, polytetrafluoroethylene (PTFE), polyvinyl chloride (PVC), or any combination thereof.

The catheter 104 allows urine to be removed from the bladder 102 via one or more openings at a second end 108 into an internal lumen and subsequently through a drainage port 120 at the first end 116 of the catheter 104. Since the second end 108 of the catheter 104 is disposed within the patient and the first end 116 of the catheter 104 is generally disposed outside of the patient, the second end may be considered a distal end 108 and the first end may be considered a proximal end 116.

In the illustrated example, the catheter 104 also includes a pair of in situ fabricated electrodes 110 (only one of which is shown in FIG. 1A for simplicity of illustration) disposed at the distal end 108 within the internal lumen used for urine drainage. For example, the fabricated electrodes 110 can be formed directly on (or over) a perimeter wall of the internal lumen of the catheter 104, with the electrodes 110 being spaced from each other along a circumferential direction of the catheter 104. In some embodiments, the electrodes 110 may cover most (e.g., ≥80%), or at least a majority, of the internal lumen wall perimeter at the distal end 108. In the illustrated example, electrical traces 114, which may also be formed directly on (or over) the perimeter wall of the internal lumen, can extend from the proximal end 116 of catheter to the respective electrode 110 in order to provide an electrical connection thereto. Alternatively, in some embodiments, the electrical traces 114 can be omitted in favor of extending the electrodes 110 substantially along an entire length of the catheter 104 from the distal end 108 to the proximal end 116.

A controller 124 can be operatively connected to the electrical traces 114 (or directly to electrodes 110, when electrical traces 114 are omitted) for applying an AC voltage thereto and measuring impedance therefrom, via respective electrical connections 122. For example, the electrical connections can include wiring coupled to pins, leads, or terminals that extend through the wall of the catheter into electrical contact with the traces 114 (or electrodes 110). Other configurations for the electrical connections are also possible. The controller 124 can include an electrical power source (e.g., AC voltage source) for applying AC voltage (e.g., at a frequency of 2 kHz or less, such as −100 Hz) to the electrodes 110. The controller 124 can also be configured to measure impedance between the electrodes 110 based on the applied AC voltage. In some embodiments, the controller 124 may be further configured to determine a biofilm status (e.g., existence and/or growth) based on the measured impedance. Alternatively or additionally, the controller 124 can send the measured impedance data to a separate processing device that determines the biofilm status.

In response to the biofilm status determination, controller 124 can communicate with a separate user interface 128 (e.g., a handheld device such as a smart phone, a bedside alert system, a nurse station, or other medical system) via a wired or wireless connection 126, for example, to generate an indication of biofilm status (e.g., an alert to a medical practitioner) or to coordinate application of remedial action (e.g., administration of antimicrobial treatments, such as an antibiotic or a quorum sensing (e.g., autoinducer-2 analog)). Further details regarding impedance measurements for biofilm detection and use of electrodes for remedial action can be found in U.S. Publication No. 2019/0060556, entitled “Systems and Methods for Detecting and Treating Bacterial Biofilms,” which is incorporated by reference herein.

FIG. 1B depicts a generalized example of a suitable computing environment 130 in which the described innovations may be implemented, such as aspects of controller 124, user interface 128, automated control of some or all of fabrication method 500, or control of voltage source 1016. The computing environment 130 is not intended to suggest any limitation as to scope of use or functionality, as the innovations may be implemented in diverse general-purpose or special-purpose computing systems. For example, the computing environment 130 can be any of a variety of computing devices (e.g., desktop computer, laptop computer, server computer, tablet computer, etc.). In some embodiments, the computing environment is an integral part of a medical system. Alternatively, in some embodiments, the computing environment is a separate system connected to the medical system, for example, by making operative electrical connections (e.g., wired or wireless) to the medical system or components thereof.

With reference to FIG. 1B, the computing environment 130 includes one or more processing units 134, 136 and memory 138, 140. In FIG. 1B, this basic configuration 150 is included within a dashed line. The processing units 134, 136 execute computer-executable instructions. A processing unit can be a general-purpose central processing unit (CPU), processor in an application-specific integrated circuit (ASIC) or any other type of processor. In a multi-processing system, multiple processing units execute computer-executable instructions to increase processing power. For example, FIG. 1B shows a central processing unit 134 as well as a graphics processing unit or co-processing unit 136. The tangible memory 138, 140 may be volatile memory (e.g., registers, cache, RAM), non-volatile memory (e.g., ROM, EEPROM, flash memory, etc.), or some combination of the two, accessible by the processing unit(s). The memory 138, 140 stores software 132 implementing one or more innovations described herein, in the form of computer-executable instructions suitable for execution by the processing unit(s).

A computing system may have additional features. For example, the computing environment 130 includes storage 160, one or more input devices 170, one or more output devices 180, and one or more communication connections 190. An interconnection mechanism (not shown) such as a bus, controller, or network interconnects the components of the computing environment 130. Typically, operating system software (not shown) provides an operating environment for other software executing in the computing environment 130, and coordinates activities of the components of the computing environment 130.

The tangible storage 160 may be removable or non-removable, and includes magnetic disks, magnetic tapes or cassettes, CD-ROMs, DVDs, or any other medium which can be used to store information in a non-transitory way, and which can be accessed within the computing environment 130. The storage 160 can store instructions for the software 132 implementing one or more innovations described herein.

The input device(s) 170 may be a touch input device such as a keyboard, mouse, pen, or trackball, a voice input device, a scanning device, or another device that provides input to the computing environment 130. The output device(s) 170 may be a display, printer, speaker, CD-writer, or another device that provides output from computing environment 130.

The communication connection(s) 190 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, radio-frequency (RF), or another carrier.

Any of the disclosed methods can be implemented as computer-executable instructions stored on one or more computer-readable storage media (e.g., one or more optical media discs, volatile memory components (such as DRAM or SRAM), or non-volatile memory components (such as flash memory or hard drives)) and executed on a computer (e.g., any commercially available computer, including smart phones or other mobile devices that include computing hardware). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, aspects of the disclosed technology can be implemented by software written in C++, Java, Perl, any other suitable programming language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means. In any of the above described examples and embodiments, provision of a request (e.g., data request), indication (e.g., data signal), instruction (e.g., control signal), or any other communication between systems, components, devices, etc. can be by generation and transmission of an appropriate electrical signal by wired or wireless connections.

Urinary Catheter with In Situ Fabricated Electrodes

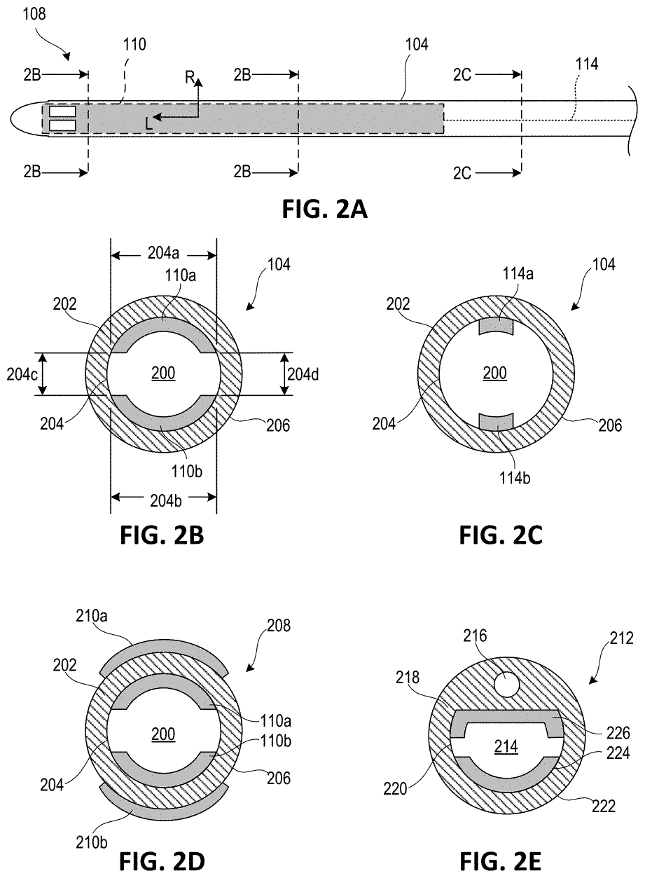

FIGS. 2A-2C illustrate an exemplary configuration of a catheter 104 with in situ fabricated electrodes 110 that can be used in the medical system of FIG. 1A or any other medical system. For example, catheter 104 can have a body or sidewall 202 spaced along a radial direction R from a central axis of the catheter. The sidewall 202 can have an internal surface 204 that defines an internal lumen 200, which extends along a longitudinal direction L from the distal end 108 to the proximal end 116 for drainage of urine from a patient's bladder. In the illustrated example of FIGS. 2A-2C, the sidewall 202 has a substantially annular configuration, thereby defining a substantially cylindrical internal lumen 200; however, other geometries are also possible according to one or more contemplated embodiments, for example, the non-circular cross-section illustrated in FIG. 2E (discussed in further detail below).

As shown in FIG. 2B, the internal surface 204 can be divided into several circumferentially extending surface regions or portions 204a-204d. Electrodes 110a, 110b can be in situ fabricated on and conforming to surface portions 204a, 204b, respectively. No electrodes are formed on surface portions 204c, 204d, thereby providing a circumferential gap between the adjacent electrodes 110a, 110b that can be used for impedance sensing. As explained in further detail below, the surface portions 204c, 204d may be screened by a patterning mold while surface portions 204a, 204b are exposed from the patterning mold, thereby allowing the electrodes 110a, 110b (or a composite layer thereof) to be formed in situ on surface portions 204a, 204b. For example, a size of the circumferential gap between adjacent electrodes 110a, 110b can be less than 1 mm (e.g., ˜300 μm), and a thickness (with respect to the radial direction R) of each electrode 110a, 110b can be less than 10 μm (e.g., ˜5 μm). In some embodiments, the width of the electrodes 110a, 110b (with respect to the circumferential direction) may cover the entirety of the perimeter of the internal surface 204 except for the surface portions 204c, 204d defining the gaps, as illustrated in FIG. 2B. Alternatively, in some embodiments, the electrodes 110a, 110b may cover at least a majority of the internal surface 204 in cross-sectional view, or even less.

In the illustrated example of FIGS. 2A-2C, the electrodes 110a, 110b extend from the distal end 108 and terminate at an intermediate region spaced from the proximal end 116 of the catheter 104. Similar to electrodes 110a, 110b, electrical traces 114a, 114b can be in situ fabricated on and conforming to surface portions of the internal surface and can extend along the longitudinal direction L to electrically connect electrodes 110a, 110b to contacts at the proximal end 116 of the catheter 104. Each electrical trace 114a, 114b can have a thickness (with respect to the radial direction R) substantially similar to that of the electrodes 110a, 110b, but a width (with respect to the circumferential direction) that is less than that of the electrodes 110a, 110b (e.g., ≤50%). Alternatively, in some embodiments, the electrodes 110a, 110b can extend to the proximal end 116 (or a region adjacent to or at least near the proximal end 116). In some embodiments, the electrical traces 114a, 114b can be omitted in favor of connecting directly to the electrodes 110a, 110b.

In some embodiments, electrodes can be provided on the exterior of the catheter in addition to the electrodes formed within the internal lumen. For example, FIG. 2D illustrates a catheter 208 with in situ fabricated electrodes 110a, 110b on respective portions of the internal surface 204 of the sidewall 202 and with external electrodes 210a, 210b provided on respective portions of an outer surface 206 of the sidewall 202. In some embodiments, the external electrodes 210a, 210b can be formed simultaneous with the internal electrodes 110a, 110b, for example, by using the same or a different patterning mold for electroless deposition of one or more metal layers. Alternatively, since the outer surface 206 of the catheter 208 is more readily accessible, the external electrodes 210a, 210b can be formed using a conventional patterning process (e.g., photolithography) followed by a metal deposition process (e.g., PVD, chemical vapor deposition (CVD), electroless deposition, etc.) prior to or after formation of the internal electrodes 110a, 110b. In some embodiments, internal electrode 110a can be electrically coupled to external electrode 210a, and internal electrode 110b can be electrically coupled to external electrode 210b. For example, the internal and external electrodes can be respectively coupled via direct electrical connection at the distal end 108 (e.g., a lead or trace extending through the sidewall 202 or through the urine drainage openings), direct electrical connection at the proximal end 116 (e.g., a common pin, lead or terminal), indirect electrical connection external to the body (e.g., via wiring prior to or within the controller 124 or separate voltage source), or in any other manner. Alternatively, in some embodiments, the external electrodes 210a, 210b are not electrically connected to and operate separately from the internal electrodes 110a, 110b.

In some embodiments, the internal lumen of the catheter in which the electrodes are formed may have a non-circular cross-section. For example, FIG. 2E illustrates a catheter 212 with in situ fabricated electrodes 224, 226 on respective portions of the internal surface 220 of the sidewall 218. The sidewall 218 of the catheter 212 may have a substantially cylindrical external surface 222; however, the internal lumen 214 may have a non-circular cross-sectional geometry in order to accommodate secondary lumen 216 (e.g., for retention balloon inflation and deflation). In the illustrated example, electrode 224 is in situ fabricated solely on an arcuate-geometry portion of the catheter internal surface 220, and thus has a configuration similar to that of electrode 110b in FIGS. 2B-2D. In contrast to electrode 110a in FIGS. 2B-2D, electrode 226 in FIG. 2E is in situ fabricated extending over and conformed to an arbitrary geometry portion of the catheter internal surface 220, for example, arcuate subportions at opposite ends of a substantially planar subportion. Alternatively, in some embodiments, each electrode can be fabricated with a first part on the arbitrary geometry portion of the catheter internal surface 220 and a second part on the arcuate geometry portion of the catheter internal surface 220. Further deviations of the cross-sectional geometry of the internal lumen 214 from a cylindrical shape may be needed to accommodate additional secondary lumens, for example, in triple lumen catheters. However, the use of electroless deposition for the in situ fabrication of the electrodes can allow the electrodes to be formed on and substantially conform to the lumen surface regardless of its cross-sectional geometry.

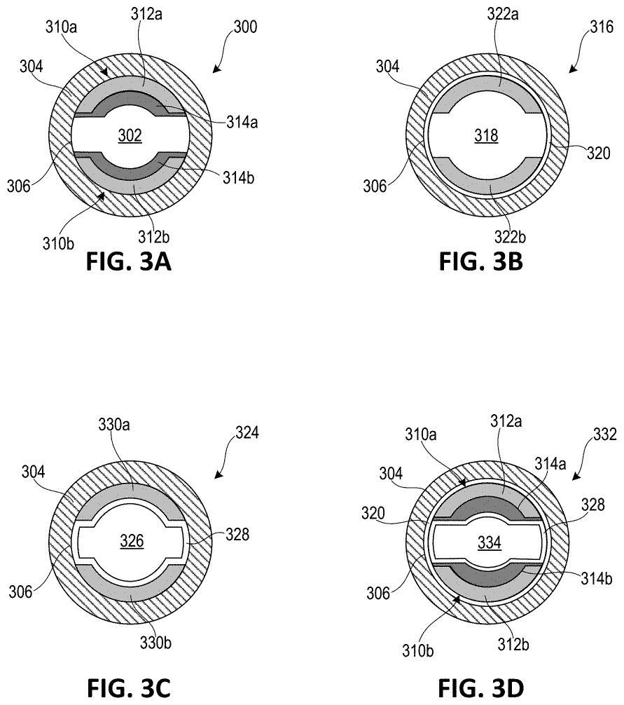

In some embodiments, one or more of the in situ fabricated electrodes can comprise multiple layers. For example, FIG. 3A illustrates a catheter 300 with multilayer electrodes 310a, 310b formed within the internal lumen 302 of the catheter. Each multilayer electrode 310a, 310a includes a base layer 312a, 312b formed in situ on and conforming to the respective portions of the internal surface 306 of sidewall 304 of the catheter. Each multilayer electrode 310a, 310b further includes a subsequent layer 314, 314b formed in situ on and conforming to the respective base metal layer 312a, 312b. Although FIG. 3A shows only two layers 312, 314, additional metal layers can be provided, either between base layer 312 and subsequent layer 314 or over subsequent layer 314, according to one or more contemplated embodiments.

Each layer 312, 314 of the electrode 310 can be formed of a metal or metal alloy, and the composition of the base layer 312 can be different from that of the respective subsequent layer 314. Alternatively or additionally, a thickness (with respect to the radial direction R) of the base layer 312 can be different from that of the respective subsequent layer 314. For example, the base layer 312 can have a thickness that is less than that of the respective subsequent layer 314. In some embodiments, the thickness of the base layer 312 is at least an order of magnitude less than that of the respective subsequent layer 314. For example, the base layer 312 can have a thickness less than 100 nm (e.g., ˜50 nm), and the subsequent layer 314 can have a thickness less than 10 μm (e.g., ˜5 μm). In some embodiments, at least the base layers 312, which is the electrode layer closest to the catheter sidewall 304 along the radial direction R, may be formed via electroless deposition. The subsequent layers 314 can be formed using the electroless deposited layer as a seed or guide layer, for example, via immersion metal, subsequent electroless deposition, or electroplating.

In some embodiments, a layer can optionally be provided over the catheter sidewall to promote adhesion of the electrode thereto. Such an adhesion promoting layer can be provided when mechanical properties of the catheter material and the electrode material are significantly different from each other. For example, if the coefficient of thermal expansion (CTE), Young's modulus, or both for a material of the catheter sidewall is more than an order of magnitude different from that of a material of the electrode (e.g., base layer 312, subsequent layer 314, or both), then the adhesion promoting layer can be provided between the catheter sidewall and the electrode to act as a buffer. In such embodiments, the adhesion promoting layer can have a CTE, Young's modulus, or both that is intermediate to that of the materials of the catheter sidewall and the electrode. Otherwise, in some embodiments, the adhesion promoting layer can be omitted.

FIG. 3B illustrates a catheter 316 with an adhesion promoting layer 320 conformally formed on an entire internal surface 306 of sidewall 304 of the catheter. Alternatively, in some embodiments, the adhesion promoting layer 320 can be formed only over select portions of the internal surface 306, for example, those surface portions where the electrodes will be formed (e.g., by using the same patterning mold for forming both the adhesion promoting layer and the electrodes). In the illustrated example, a pair of electrodes 322a, 322b are formed in situ within the internal lumen 318, in particular, on and conforming to respective surface portions of the adhesion promoting layer 320. In some embodiments, the adhesion promoting layer 320 can be formed of a polymer, such as parylene C. In some embodiments, a thickness (with respect to the radial direction R) of the adhesion promoting layer 320 can be greater than that of the respective electrode 322. For example, the adhesion promoting layer 320 can have a thickness less than 50 ηm (e.g., ≤25 μm), and the electrode 322 can have a thickness less than 10 μm (e.g., ˜5 μm). Alternatively, in some embodiments, a thickness of the adhesion promoting layer 320 may be the same as or less than that of the respective electrode 322.

In some embodiments, a layer can optionally be provided over the electrodes to protect the electrodes from the surrounding environment or vice versa. Such a protection layer can be provided when there is a concern that the electrode material may degrade over time and/or that ions may leach from the electrodes into the surrounding environment (e.g., in vivo). Otherwise, in some embodiments, the protection layer can be omitted. FIG. 3C illustrates a catheter 324 with a protection layer 328 conformally formed on exposed surfaces within the internal lumen 326 of the catheter. For example, the protection layer 328 can be formed on the exposed surfaces of the pair of in situ fabricated electrodes 330a, 330b, as well as on the exposed surface portions of the internal surface 306 of catheter sidewall 304 between the electrodes 330a, 330b. Alternatively, in some embodiments, the protection layer 328 can be formed only over select portions within the internal lumen 326, for example, exposed surfaces of the electrodes 330 (e.g., by using the same patterning mold for forming both the adhesion promoting layer and the electrodes). In some embodiments, the protection layer 328 can be formed for a polymer, such as parylene C. Alternatively or additionally, a thickness (with respect to the radial direction R) of the protection layer 328 can be greater than that of the electrodes 330. For example, the protection layer can have a thickness less than 2 μm (e.g., ≤1 μm), and the electrodes 330 can have a thickness less than 10 μm (e.g., ˜5 μm).

In some embodiments, a catheter can have electrodes comprised of multiple layers formed on an adhesion protection layer and a protective layer covering the electrodes. For example, FIG. 3D illustrates a catheter 332 that incorporates the features of FIGS. 3A-3C, in particular, the multilayer electrodes 310, the adhesion promoting layer 320, and the protection layer 328 within internal lumen 334. In some embodiments, the material for the adhesion promoting layer 320 can be the same as that of the protection layer 328 (e.g., both formed of parylene C).

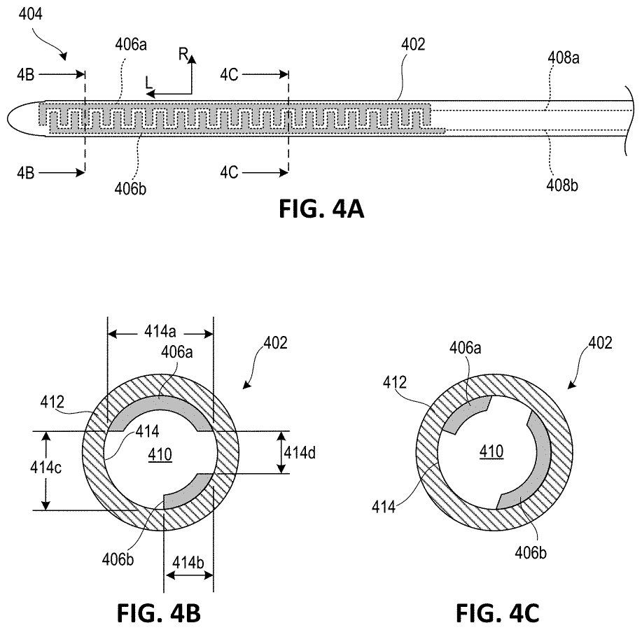

Although FIGS. 2A-3D illustrate relatively simple electrode patterns, embodiments of the disclosed subject matter are not limited thereto. Rather, more complex electrode patterns, such as an interdigitated or comb finger pattern, are also possible for the in situ fabricated electrodes by appropriate design of the patterning mold. For example, FIGS. 4A-4C illustrate an exemplary configuration of a catheter 402 with in situ fabricated electrodes 406a, 406b having an interdigitated electrode pattern. For example, catheter 402 can have a sidewall 412 with an internal surface 414 that defines an internal lumen 410, which extends along a longitudinal direction L from the distal end 404 to the proximal end for drainage of urine from a patient's bladder. As shown in FIG. 4B, the internal surface 414 can be divided into several circumferentially extending surface portions 414a-414d. Electrodes 406a, 406b can be in situ fabricated on and conforming to surface portions 414a, 414b, respectively. No electrodes are formed on surface portions 414c, 414d. In some embodiments, the circumferential gap over surface portion 414d between the tip edge of electrodes 406a and the recessed edge of electrode 406b surface portion (or vice versa) and/or a longitudinal gap between side edges of adjacent fingers of the interdigitated electrodes 406a, 406b can be used for impedance sensing. For example, a size of the surface portion 414d defining the gap between the interdigitated electrodes 406a, 406b can be less than 1 mm (e.g., ˜300 μm). In some embodiments, the surface portion 414c may be significantly larger than the surface portion 414d. Alternatively or additionally, surface portion 414c can be patterned similar to surface portion 414d, so as to fabricate electrodes 406a, 406b as double-ended interdigitated electrodes.

In some embodiments, the electrodes (e.g., any of 110, 210, 224, 226, 312, 314, 322, 330, and 406 in FIGS. 2A-4C) and/or electrical traces (e.g., any of 114 and 408 in FIGS. 2A-4C) can be composed of one or more metals or metal alloys, such as, but not limited to, tin, palladium, nickel, gold, titanium, silver, zinc, copper, chromium, platinum, or combinations thereof. In some embodiments, the electrodes (e.g., any of 110, 210, 224, 226, 312, 314, 322, 330, and 406 in FIGS. 2A-4C) and/or electrical traces (e.g., any of 114 and 408 in FIGS. 2A-4C) can include non-metal materials in addition to the metal or metal alloys, either within the metal or metal alloy layer or as a separate layer.

Although only two electrodes are shown within the internal lumen in FIGS. 2B-4C, any number of in situ fabricated metal layers (which may or may not be configured as electrodes) can be formed within the internal lumen of the catheter. For example, in some embodiments, at least two pairs of electrodes, which may be interconnected for integrated operation or isolated for separate operation, can be formed within the internal lumen. Similarly, although only two electrodes are shown on the external surface in FIG. 2D, any number of metal layers (which may or may not be configured for operation as electrodes) can be formed on the external surface of the catheter.

### Method for In Situ Fabrication of Electrodes

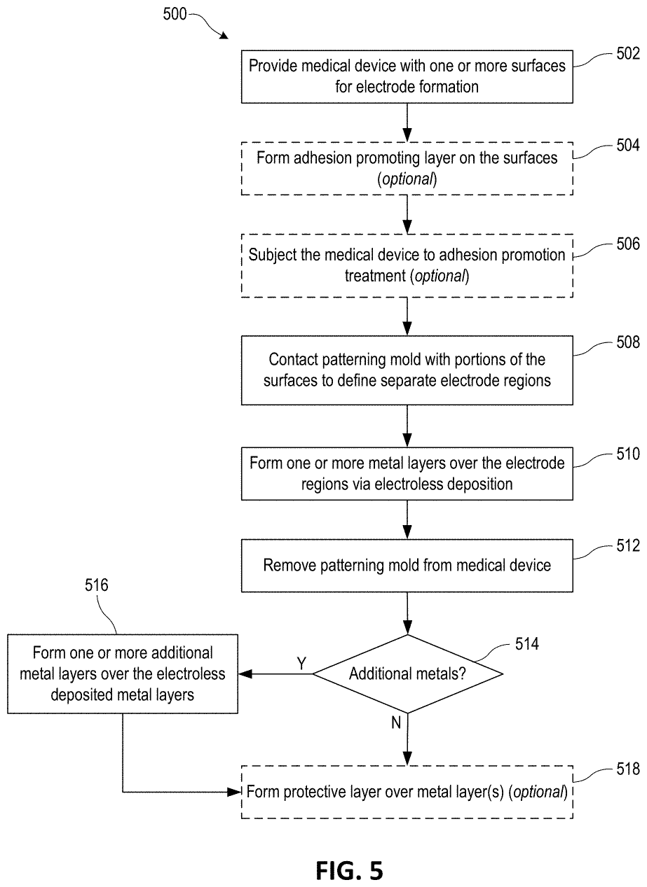

FIG. 5 illustrates an exemplary method 500 for in situ fabrication of sensor electrodes on or over surface portions of a medical device or system. The method 500 can initiate at process block 502, were a previously fabricated medical device or system can be provided. In some embodiments, the medical device or system has a non-planar surface (e.g., curved or piecewise curved) on or over which one or more metal layers are to be formed for use as sensor electrodes. Alternatively or additionally, in some embodiments, the surface of the medical device or system on or over which the one or metal layers are to be formed may be an internal surface, which may otherwise be difficult to patterning using conventional photolithographic techniques.

The method 500 can proceed to optional process block 504, where an adhesion promoting layer can be formed, if desired. For example, an adhesion promoting layer may be provided if the material of the medical device has a CTE, Young's modulus, or both that differs from the material of the metal layer to be formed thereon or thereover by more than an order of magnitude. The adhesion promoting layer can have a CTE, Young's modulus, or both that is intermediate to that of the materials of the medical device and the metal layer for the sensor electrodes. In some embodiments, the adhesion promoting layer can be a polymer conformally applied to an entire internal surface of the medical device (e.g., an entire circumference of an internal sidewall defining a catheter lumen) or to only a portion of the internal surface where the sensor electrode metal will be formed. In some embodiments, the adhesion promoting layer can be formed of parylene C.

The method 500 can proceed to optional process block 506, where an adhesion promotion treatment can be performed, if desired. In some embodiments, the adhesion promoting layer (if provided) and/or to the underlying surface of the medical device is subject to the adhesion promotion treatment. For example, the adhesion promotion treatment can be effective to increase the surface reactivity (e.g., by introducing reactive carboxylic groups thereto) and/or to clean the surface of organic material. In some embodiments, the adhesion promotion treatment can comprise oxygen plasma and/or ultraviolet (UV) ozone. Alternatively or additionally, in some embodiments, the adhesion promotion treatment can comprise a chemical agent applied to the adhesion promoting layer and/or the underlying surface of the medical device. For example, the chemical agent can be one or more commercially-available adhesion promotors or coupling agents (e.g., organosilane coupling agents).

The method 500 can proceed to process block 508, where a patterning mold is placed in contact with surface portions of the medical device to be screened during subsequent metal layer formation. For example, the patterning mold can have protruding portions, which contact the surface portions of the medical device where metal layer formation is not desired, and recessed portions, which are spaced from the surface of the medical device to define separate electrode formation regions. In some embodiments, the patterning mold can be disposed within an internal lumen of the medical device (e.g., by axially inserting the patterning mold into a proximal end of a catheter). In some embodiments, the patterning mold can be reconfigurable (e.g., radially expandable), for example, between a first configuration contacting the medical device surface portions and a second configuration spaced from the same medical device surface portions. Such reconfigurability may add in the insertion and removal of the patterning mold into the medical device while minimizing damage to previously formed structures (e.g., deposited metal layers) therein. In some embodiments, the patterning mold can be composed of a polymer (e.g., 3D printed resin), sacrificial material (e.g., silicon, silicon dioxide, etc.), deformable material (e.g., polycaprolactone), or any combination thereof.

The method 500 can proceed to process block 510, where a first metal layer can be in situ formed over the electrode formation regions defined by the patterning mold using electroless deposition. A metal layer can thus be formed on the surface portions exposed from the patterning mold while the surface portions in contact with the patterning mold are prevented from having any metal deposited thereon. In some embodiments, the electroless deposition process can be a single step or multi-step process. For example, electroless deposition of nickel as the electrode layer (or part thereof) can comprise a sensitization process to bond tin ions to the exposed surface portions using stannous chloride solution (in methanol and water) and trichloroacetic acid, followed by a substitution process to replace tin ions on the surface with palladium using a sodium tetrachloropalladate solution, and finally formation of a nickel layer on with the palladium serving as a nucleation site using a nickel electroless plating bath. Other metal electroless deposition processes are also possible according to one or more embodiments, such as, but not limited to, electroless nickel immersion gold (ENIG) plating, electroless nickel electroless palladium immersion gold (ENEPIG) plating, electroless nickel-phosphorus plating, electroless copper plating, electroless palladium plating, or any combination thereof. In some embodiments, process block 510 can comprise repeating the electroless deposition more than once with the same or different metals, for example, to form each electrode as a multilayer stack.

The method 500 can proceed to process block 512, where the patterning mold can be removed from the medical device. In some embodiments, the patterning mold can be removed from the medical device, for example, by axially retracting the patterning mold from the proximal end of a catheter. In some embodiment, the patterning mold can be formed of a soft or flexible material (e.g., a 3D-printed resin) such that the patterning mold can be removed without damaging the electroless deposited metal. Alternatively or additionally, some or all of the patterning mold can be formed to be sacrificial. In such embodiments, the removing of process block 512 can include melting, etching, dissolving, or deforming the sacrificial portions such that the patterning mold does not contact the internal lumen of the catheter. For example, part or all of the patterning mold can be formed of silicon (removed in situ xenon difluoride etching), silicon dioxide (removed in situ by hydrofluoric acid etching), polycaprolactone (having a relatively low melting temperature, and therefore can be deformed away from walls with or without heating), or wax (removed in situ by heating to melt or at least deformed away from walls with or without heating). Other materials and sacrificial removal techniques for the patterning mold are also possible according to one or more contemplated embodiments.

In some embodiments, the patterning mold can be reconfigured to the second configuration where the patterning mold no longer contacts the medical device surface portions. For example, the patterning mold can have a radially expanding portion (e.g., employing a Hoberman mechanism or similar) to transition between a contacting state for electroless deposition in process block 510 and a non-contacting state for removal in process block 512. Alternatively or additionally, the patterning mold can be formed of multiple parts that can be separately removed. For example, in an assembled state of the multiple parts, the patterning mold may be in contact with the surface portions of the medical device, but after removal of one or more of the parts, the remaining parts of the patterning mold can be spaced from the surface portions.

The method 500 can proceed to decision block 514, where it is determined if additional metal layers are desired to form the electrodes. In some embodiments, the electroless deposited metal layers of process block 510 are sufficient to form the electrodes, in which case the method 500 can proceed to optional process block 518. Otherwise, if additional metal layers are desired, the method 500 and proceed to process block 516, wherein one or more additional layers are formed on or over the electroless deposited layers. In some embodiments, the additional metal layers can also be formed using electroless deposition but using a different metal. Alternatively, in some embodiments, the additional metal layers can be formed using immersion metal or electroplating. When electroplating is used, process block 516 can include making electrical contact to the electroless deposited metal layers (e.g., by clipping to exposed portions of the metal layers or by a separate mold inserted into the medical device to make electrical contact with the metal layers). In some embodiments, the electroless deposited metal layers can serve as a seed layer for the subsequent electroplating. For example, the electroplating may be performed by immersing the medical device within transene sulfite gold (TSG) electroplating solution and cycling an applied voltage between 0 and −0.5 V for 100 cycles at 25 mV/s to yield an electroplated gold layer (e.g., having a thickness of ˜5 μm).

After process block 516 or if no additional metal layers were desired at decision block 514, the method can proceed to optional process block 518, where a protective layer can be formed, if desired. In some embodiments, the protective layer can be a polymer conformally applied to an entire surface of the medical device (e.g., an entire circumference of an internal sidewall defining the catheter lumen) or to only a portion of the internal surface where the sensor electrode metal was formed (e.g., by retaining the patterning mold in place until after the protective layer is formed). In some embodiments, the material type and thickness of the protective layer is selected so as to not substantially affect sensor performance (e.g., impedance measurements). For example, the protective layer can formed of parylene C and have a thickness of 1 μm or less.

Although not explicitly discussed, prior to each or any of the above noted process blocks, the medical device or a portion thereof may be cleaned in preparation for the subsequent process block, for example, by sequentially rinsing with acetone, methanol, and isopropanol. Although some of blocks 502-518 of method 500 have been described as being performed once, in some embodiments, multiple repetitions of a particular process block may be employed before proceeding to the next decision block or process block. In addition, although blocks 502-518 of method 500 have been separately illustrated and described, in some embodiments, process blocks may be combined and performed together (simultaneously or sequentially). Moreover, although FIG. 5 illustrates a particular order for blocks 502-518, embodiments of the disclosed subject matter are not limited thereto. Indeed, in certain embodiments, the blocks may occur in a different order than illustrated or simultaneously with other blocks. For example, the illustrated order of some of the above noted process blocks can be altered such that the patterning mold is in place for one or more of the adhesion promoting layer formation (process block 504), adhesion promotion treatment (process block 506), electroplating (process block 516), and protective layer formation (process block 518). Alternatively or additionally, in some embodiments, at least an initial substep of the electroless deposition of process block 510 (e.g., sensitization) can occur prior to the patterning mold being put into place in process block 508.

### Patterning Molds for In Situ Fabrication of Electrodes

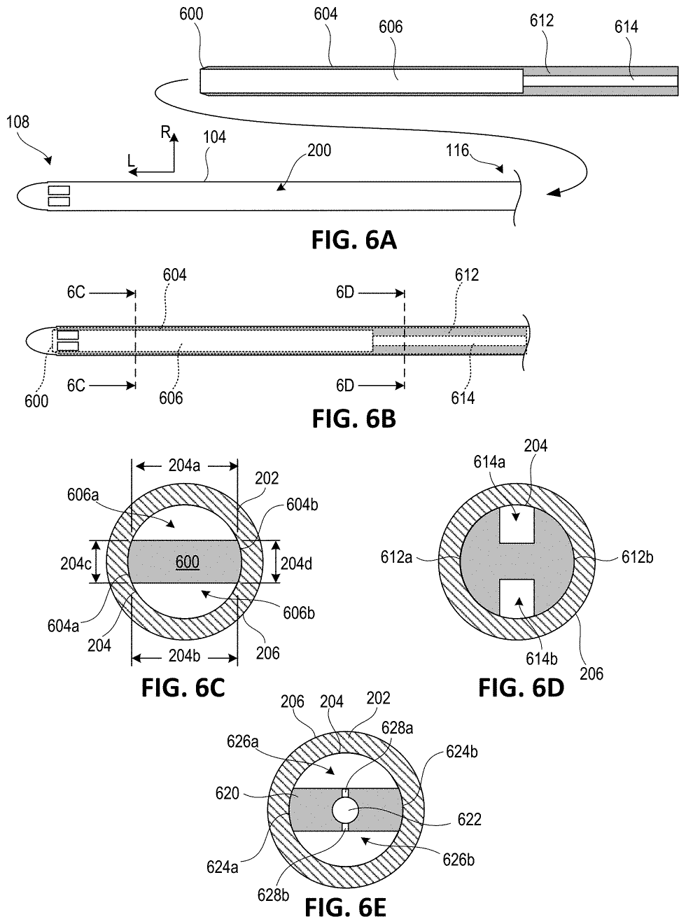

FIGS. 6A-6D illustrate an exemplary assembly of a catheter 104 and patterning mold 600 (also referred to as patterning scaffold, screening scaffold, screening mold, or electrode pattern template) for in situ fabrication of patterned electrodes within the internal lumen 200 of the catheter. As discussed above, the internal surface 204 of the catheter 104 can be divided into several circumferentially extending surface portions 204a-204d. Electrodes 110a, 110b will be formed on surface portions 204a, 204b, respectively, while no electrodes will be formed on surface portions 204c, 204d. Once inserted into the internal lumen 200, the patterning mold 600 has protruding regions or contact portions 604a, 604b that contact the surface portions 204c, 204d, respectively. The patterning mold 600 also has spaced regions or recessed portions 606a, 606b that are spaced from surface portions 204a, 204b, respectively. When the catheter 104 includes in situ fabricated electrical traces 114 for electrically connecting to the in situ fabricated electrodes 110, the patterning mold 600 can additionally include separate protruding regions 614a, 614b and spaced regions 612a, 612b for forming the electrical traces. Accordingly, when the assembly is subject to an electroless deposition process, the solution is able to infiltrate the spaced regions 606, 614 in order to deposit a metal layer on the exposed surface portions (e.g., surface portions 204a, 204b). Meanwhile, the tight contact between protruding regions 604, 612 and the facing surface portions of the catheter inner surface 204 (e.g., surface portions 204c, 204d) prevent the electroless deposition solution from infiltrating therebetween, such that these surface portions remain substantially metal free.

In some embodiments, the patterning mold can optionally include one or more internal conduits that fluidically connect to ports that open to the spaced regions. For example, FIG. 6E illustrates an assembly of a catheter and a patterning mold 620 that has a central conduit 622 that extends along the longitudinal direction L. The central conduit 622 can be fluidically connected to ports 628a, 628b, which are respectively connected to spaced regions 626a, 626b. In some embodiments, the central conduit 622 can be used to deliver solution (e.g., electroless deposition solution, electroplating solution, etc.) to the spaced regions 626a, 626b defined within the internal lumen of the catheter. Alternatively or additionally, the central conduit 622 can be used to remove air trapped within spaced regions 626a, 626b during delivery of solution (e.g., electroless deposition solution, electroplating solution, etc.) to the internal lumen of the catheter.

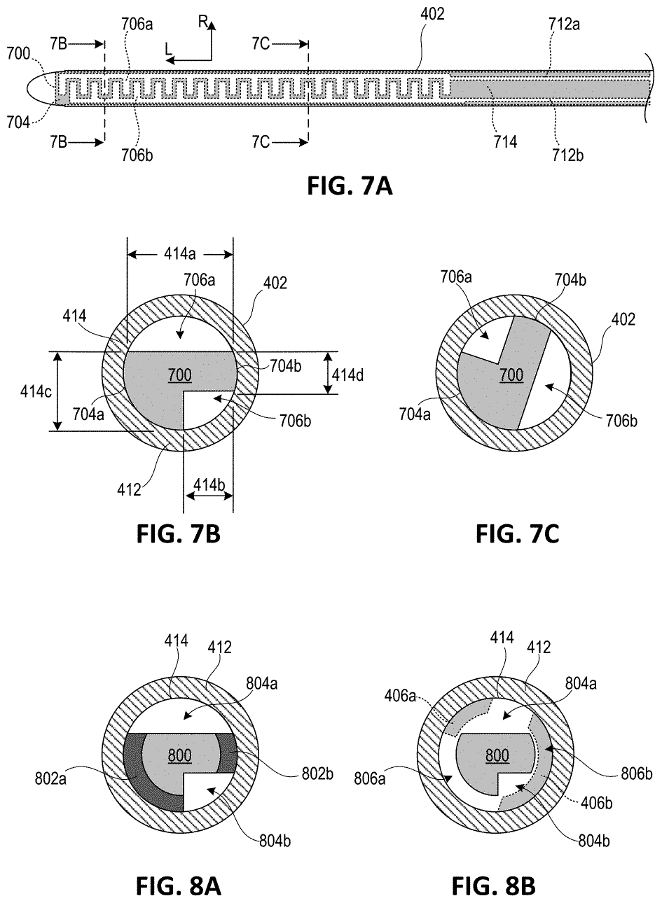

FIGS. 7A-7C illustrate another exemplary assembly of a catheter 402 and patterning mold 700 for in situ fabrication of an interdigitated patterned electrodes within the internal lumen of the catheter. As discussed above, the internal surface 414 of the catheter 402 can be divided into several circumferentially extending surface portions 414a-414d. Interdigitated electrodes 406a, 406b will be formed on surface portions 414a, 414b, respectively, while no electrodes will be formed on surface portions 414c, 414d. Once inserted into the internal lumen, the patterning mold 700 has protruding regions 704a, 704b that contact the surface portions 714c, 714d, respectively. The patterning mold 700 also has spaced regions 706a, 706b that are spaced from surface portions 414a, 414b, respectively. When the catheter 402 includes in situ fabricated electrical traces 408 for electrically connecting to the in situ fabricated electrodes 406, the patterning mold 700 can additionally include separate protruding regions 714 and spaced regions 712 for forming the electrical traces. Accordingly, when the assembly is subject to an electroless deposition process, the solution is able to infiltrate the spaced regions 706, 712 in order to deposit a metal layer on the exposed surface portions (e.g., surface portions 414a, 414b). Meanwhile, the tight contact between protruding regions 704, 714 and the facing surface portions of the catheter inner surface 414 (e.g., surface portions 414c, 414d) prevent the electroless deposition solution from infiltrating therebetween, such that these surface portions remain substantially metal free. As is apparent from a comparison of FIGS. 7B and 7C, the location of the recessed regions 706a, 706b (and the corresponding electrode layers formed therein) with respect to the circumferential direction changes depending on the location along the longitudinal direction. Thus, the geometry of the interdigitated electrodes 406 may pose a challenge in removing the patterning mold 700 from the catheter 402, since the patterning mold 700 may have a protruding region 704a or 704b that would impact a formed electrode as the patterning mold 700 is retracted proximally. Accordingly, the patterning mold can be adapted to disengage from contacting the inner surface of the catheter prior to removal.

In some embodiments, the patterning mold can be formed with one or more sacrificial portions. For example, FIGS. 8A-8B illustrate an assembly of catheter 402 and a patterning mold 800 having a sacrificial outer contact regions 802a, 802b. During electroless deposition (or other patterning steps), the contact regions 802a, 802b contact the inner surface 414 of the catheter to prevent layer formation thereon, while layers can be formed on the surface portions exposed by spaced regions 804a, 804b. However, once patterning is no longer needed, the contact regions 802a, 802b can be removed (e.g., by melting, dissolving, etching, etc. to define additional spaced regions 806a, 806b), such that the remaining patterning mold 800 can be removed by retracting proximally along the longitudinal direction without impacting interdigitated electrodes 406a, 406b formed at other axial locations.

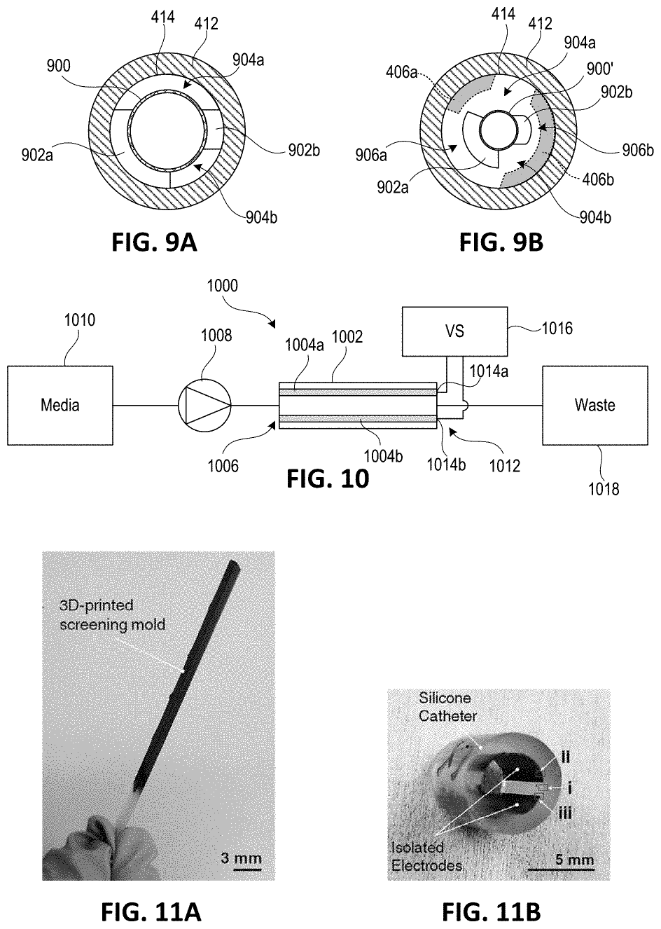

Alternatively or additionally, in some embodiments, the patterning mold can be reconfigurable between contacting and non-contacting states. For example, FIGS. 9A-9B illustrate an assembly of catheter 402 and a patterning mold that can radially expand and collapse. During electroless deposition (or other patterning steps), a reconfigurable frame 900 is radially expanded such that contact regions 902a, 902b contact the inner surface 414 of the catheter to prevent layer formation thereon while layers can be formed on the surface portions exposed by spaced regions 904a, 904b. For example, the reconfigurable frame 900 can employ a Hoberman mechanism or another mechanism that translates linear motion along the longitudinal direction L into radial expansion/contraction of frame 900. Once patterning is no longer needed, the reconfigurable frame adopts a collapsed configuration 900′, such contact regions 902a, 902 are spaced from the inner surface 414 to form additional spaced regions 906a, 906b. The patterning mold can then be removed by retracting proximally along the longitudinal direction without impacting interdigitated electrodes 406a, 406b formed at other axial locations.

Although the patterning mold illustrated in FIGS. 6A-9B is configured for forming only two electrodes within the internal lumen, the patterning mold can be configured to form any number of in situ fabricated metal layers (which may or may not be configured as electrodes) within the internal lumen of the catheter. For example, in some embodiments, the patterning mold can be configured to form at least two pairs of electrodes, which may be interconnected for integrated operation or isolated for separate operation, within the internal lumen. Similarly, although only two electrodes are shown on the external surface in FIG. 2D, any number of metal layers (which may or may not be configured for operation as electrodes) can be formed on the external surface of the catheter.

### Fabricated Examples and Experimental Results

Gold impedance sensor electrodes were fabricated directly on a 22 Fr elastomeric Foley catheter by an in situ fabrication process. First, the catheter was coated in a layer of parylene-C (e.g., ˜25 μm-thick), which may help reduce the mechanical mismatch (e.g., CTE, Young's modulus, etc.) between the plated metal and the silicone polymer substrate and/or help improve adhesion. Without the parylene-C layer, cracks may appear in the metal, thereby resulting in a significant loss of conductivity. The catheter was then cleaned using acetone, methanol, and isopropanol. The catheter was then subjected to oxygen plasma treatment (200 W, 1 min.) in order to introduce reactive carboxylic groups to the surface of the parylene-C, for example, to improve the adhesion of the subsequently deposited electrodes. Immediately after the oxygen plasma treatment, the catheter was immersed in 0.026 M stannous chloride solution (in 1:1 methanol and water) with 0.07 M trichloroacetic acid for 45 minutes. This sensitization process bonds tin ions to the catheter surface.

After the sensitization process, the catheter was rinsed with methanol, and a patterning mold (e.g., a 3D-printed mold) was subsequently inserted into the catheter. The 3D-printed mold screened portions of the inner lumen of the catheter from the electroless plating solutions, thereby producing separate electrodes. The mold was fabricated using a FormLabs Form 2 SLA 3D printer (FormLabs Inc., Somerville, Mass.) with photopolymer resin. 3D printing dramatically simplifies the electrode patterning compared to techniques like photolithography, particularly in hard to reach areas like the catheter lumen. However, other techniques for forming the patterning mold are also possible.

After insertion of the mold, the catheter was filled with a 10 mM sodium tetrachloropalladate solution for 5 hours, which replaced the tin on the surface with palladium. Palladium served as a nucleation site for the formation of the subsequent nickel seed layer. In particular, the catheter was immersed for 45 seconds in a nickel electroless plating bath, which generated a thin nickel layer (e.g., ≤50 nm) on the inner lumen of the catheter. The 3D-printed mold was then removed for subsequent gold electroplating. In particular, electrical connection was made to the nickel layer, and the catheter was immersed in transene sulfite gold (TSG-250) electroplating solution. Electroplating voltage was then cycled from 0 to −0.5 V for 100 cycles at a scan rate of 25 mV/s in order to form on a gold layer (e.g., ≤1 μm) atop the nickel seed layer. The resulting gold electrodes were then coated with another layer of parylene-C (e.g., ˜1-μm thick) to prevent ions from leaching from the electrodes.

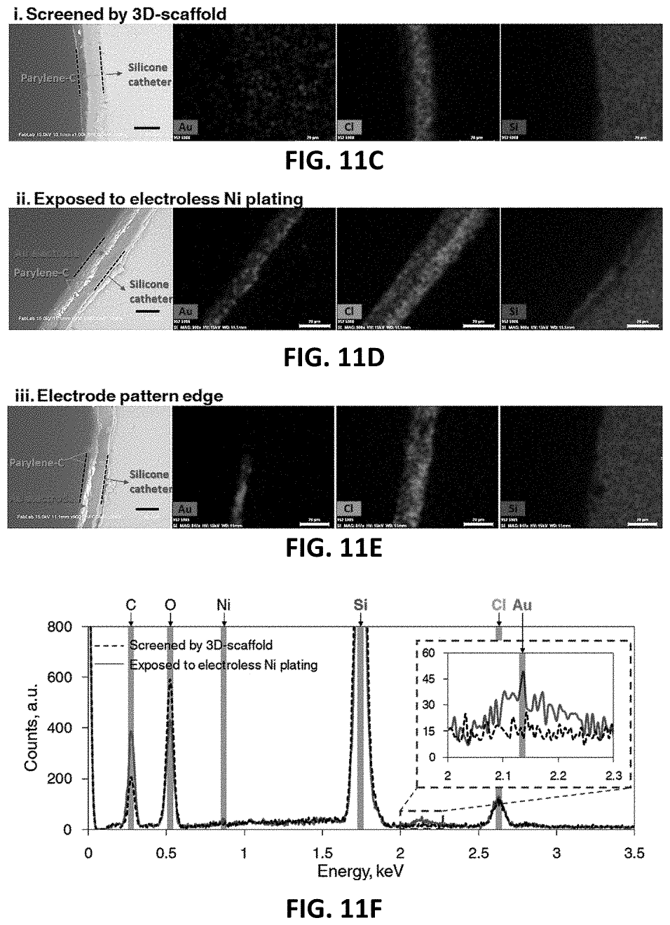

The sensor electrodes patterned on the inner lumen of the catheter are shown in the cross-section image of the catheter section in FIG. 11B. As shown in FIG. 11A, the patterning mold used in this example had a substantially rectangular cross-section with outer rounded edges that contact the catheter lumen to screen the lumen wall portions for selective electroless plating. The dark portions of the mold in FIG. 11A have nickel plated thereon from the electroless deposition process. Note that more complex electrodes with larger sensor interfaces could be produced using more complex 3D printed molds, such as interdigitated patterns.

Scanning electron microscopy (SEM) and energy-dispersive X-ray spectroscopy (EDS) analysis were used to examine the cross section of the fabricated catheter device. FIGS. 11C-11E show cross-sectional SEM images of different portions (i)-(iii) (see FIG. 11B) of the electrode-patterned catheter with corresponding EDS scans. In the SEM images, the silicone, metal, and the two parylene-C layers are distinctly shown. The EDS analysis further confirms the presence of these layers by means of their signature elements, in particular, gold at 2.120 keV (for the electrodes), chlorine at 2.622 keV (for the parylene-C layers), and silicon at 1.749 keV (for the silicone wall of the catheter). A significant difference between the screened area (shown in FIG. 11c) and the exposed area (FIG. 11D) is that the screened area lacks any metal layer, as also reflected in the inset of FIG. 11F. As shown in FIG. 11E, the well-defined electrode pattern edge clearly indicates the success in use of the 3D printed screening mold for patterning of in situ electroless plating on curved confined surfaces.

Catheters with in situ fabricated electrodes were then subjected to biofilm testing using the experimental setup 1000 illustrated in FIG. 10. In particular, catheter section 1002 with plated electrodes 1004a, 1004b was interfaced with flexible polymer tubing (e.g., Tygon® tubing) to form a flow system for introducing fresh Luria broth (LB) growth media (e.g., from reservoir 1010) and bacterial cells (e.g., injected into the tubing connected to the inlet end 1006). Electrical connections 1014a, 1014b were made using 24-gauge wire clipped to the two electrodes 1004a, 1004b at the outlet end 1012 of the catheter section 1002. The electrical connections and tubing were sealed using epoxy. A peristaltic pump 1008 was used to drive flow at 7 ml/h through the system from the LB media reservoir 1010 through the catheter 1002 with the sensor to a waste reservoir 1018. Prior to initiation of each experiment, the media 1010 and waste 1018 reservoirs were sterilized in an autoclave at 121° C., and the catheter 1002 and tubing were sterilized by flushing with ethanol and UV light exposure for an hour. The entire setup 1000 was assembled in a sterile biosafety cabinet.

Escherichia coli K12 W3110 were cultured overnight in a 5 ml culture tube in an incubator shaker at 250 rpm and 37° C. The bacterial culture was then diluted to an OD600 of 0.25 and added to the system via syringe. The bacteria were allowed to attach for 2 hours to the electrode surfaces under a no-flow condition. Then, LB media was flowed for 24 hours as the biofilm grows. No bacterial cells were added in the control samples. Impedance spectra were gathered using an electrochemical workstation (CH Instruments, Inc., Austin, Tex.) from 10¹-10⁶ Hz at a 50 mV amplitude. These spectra were gathered before the introduction of bacteria with LB media only, at the beginning of the 24-hour biofilm growth period, at the end of the biofilm growth period, and after the growth period after cleaning with ethanol. In addition, the impedance was monitored in real-time at 100 Hz throughout the growth period.

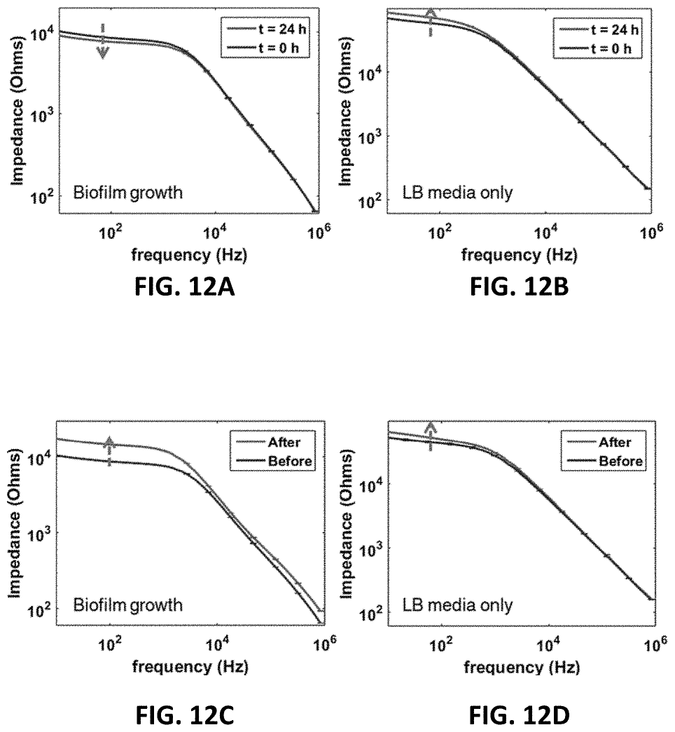

As shown in FIG. 12A, impedance spectroscopy at the beginning and after 24-hours of biofilm growth displayed a frequency-dependent decrease in impedance associated with biofilm growth. This was consistent with previous reports indicating that the capacitive component of the impedance changes with biofilm formation due to the accumulation of charged proteins and metabolites. In contrast, control samples without any biofilm showed a frequency-independent increase in impedance, as shown in FIG. 12B. Without being bound by any particular theory, it is believed that the increase in impedance is related to electrode degradation, potentially caused by the dissolution of metal ions due to the applied voltage in an electrolyte media. This is further evidenced by impedance spectroscopy of the sensor electrodes which had been used to detect biofilm before the cells were added and after cleaning with ethanol. As shown in FIG. 12C, the electrodes displayed a frequency-independent increase in impedance when measuring the impedance in LB media after the electrodes had been cleaned of biofilm. A similar trend can be seen in the control electrodes before and after the growth period, as shown in FIG. 12D. The increase in impedance after the removal of biofilm with ethanol reinforces the role of biofilm in the decrease in impedance seen with the sensor.

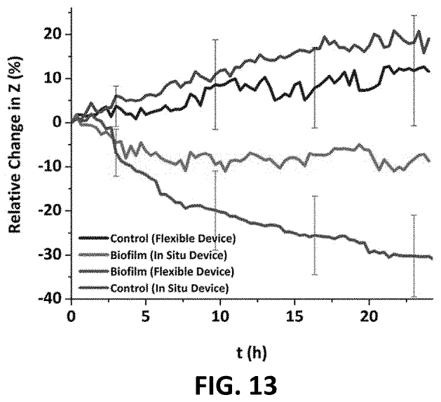

The in situ patterned electrodes were further utilized for continuous, real-time monitoring of biofilm formation, which can allow a continuous readout of the state of the catheter surface and thereby provide a more effective tool for biofilm infection management. An AC frequency of 100 Hz was selected for biofilm monitoring, since it was in the middle of the frequency range that displayed an impedance decrease in FIG. 12A. However, other AC frequencies may also be selected according to one or more contemplated embodiments. The impedance was recorded continuously for samples with and without biofilm over 24 hours. As shown in FIG. 13, the samples with biofilm showed a sharp decrease over the first 7 hours, followed by a plateau of relatively little change over the next 17 hours. In total, the impedance decreased approximately 10% over the 24-hour period. This is consistent with the growth dynamics of bacterial biofilm, with a rapid growth phase followed by a stable mature biofilm phase. In contrast, the control sample without any bacterial cells showed an increase in impedance of approximately 20%. This increase was relatively steady throughout the 24-hour period, presumably due to electrode degradation, as discussed above. The results for the catheter with in situ fabricated electrodes were also compared to continuous biofilm impedance sensing results obtained using a separate flexible impedance sensor coupled to a urinary catheter. The flexible impedance sensor was fabricated monolithically using photolithography, but then must be rolled and inserted into the catheter. Both the control and biofilm experiments for the catheter with flexible impedance sensor showed similar performance to the catheter with in situ fabricated electrodes.

### Conclusion

Although the examples and discussion above has focused primarily on urinary catheters, the teachings disclosed herein can be readily applied to other medical devices, such as, but not limited to, other types of catheters or indwelling medical devices (e.g., coronary catheter, central venous catheter, Quinton catheter, or any other type of vascular access device; hypodermic needle, Tuohy needle, or any other type of medical needle; endotracheal tube, tracheostomy tube, enteral feeding tube, or any other type of medical tubing; wound drain, external ventricular drain, surgical drain, or any other type of medical drain, conduit, or cannula; dental implant, orthopedic implant, coronary/heart valve, or any other type of medical implant. Other systems or devices may also benefit from the disclosed in situ fabrication techniques, for example, in systems or devices where conventional metal patterning techniques (e.g., photolithography) may be difficult or impossible to employ. Accordingly, embodiments of the disclosed subject matter are not limited to the specific examples described herein.

Moreover, although the examples and discussion above has focused primarily on the detection of bacterial biofilm via changes in impedance, embodiments of the disclosed subject matter are not limited thereto. Rather, the in situ fabricated electrodes can be used for any other purpose, such as but not limited to applying AC voltages for biofilm treatment or mitigation (e.g., to enhance efficacy of an administered antimicrobial agent via a bioelectric effect); heating; capacitive sensing; evaluating response of a host tissue to a prosthetic implant; providing an identification or decoration; or for any other purpose.

In addition, although reference has been made herein to sensing within a patient, embodiments of the disclosed subject matter are not limited to use in a human. Indeed, embodiments of the disclosed subject matter can find wide application to non-human in vivo environments (e.g., animal) or any other environment where monitoring and/or treating bacterial growth may be desirable (e.g., benchtop testing setups for studying biofilm growth).

Any of the features illustrated or described with respect to FIGS. 1A-13 can be combined with any other features illustrated or described with respect to FIGS. 1A-13 to provide systems, methods, devices, and embodiments not otherwise illustrated or specifically described herein. For example, the external electrodes illustrated in FIG. 2D can be used in conjunction with the internal electrodes illustrated in any of FIGS. 2B-2C, 2E, 3A-3D, and 4B-4C. In another example, the multi-layer electrode configuration of FIG. 3A can be used instead of the single layer electrode configurations of FIGS. 2B-2E, 3B-3C, and 4B-4C. In still another example, the adhesion promoting layer of FIG. 3B, the protection layer of FIG. 3C, or both can be applied to the electrode configurations of any of FIGS. 2B-2E, 3A-3C, and 4B-4C. In yet another example, the catheters of any of FIGS. 2B-2D, 3B-3C, 4B-4C, 6C-6E, and 7B-9B could have the multi-lumen cross-sectional configuration illustrated in FIG. 2E. One of ordinary skill in the art will readily appreciate additional examples. All features described herein are independent of one another and, except where structurally impossible, can be used in combination with any other feature described herein.

In view of the many possible embodiments to which the principles of the disclosed technology may be applied, it should be recognized that the illustrated embodiments are only preferred examples and should not be taken as limiting the scope of the disclosed technology. Rather, the scope is defined by the following claims. We therefore claim all that comes within the scope and spirit of these claims.

1. A method of fabricating a catheter with in situ sensor electrodes, the catheter having an internal lumen extending between first and second longitudinal ends of the catheter, the method comprising:
inserting a patterning mold into the internal lumen via the first longitudinal end of the catheter such that first and second surface portions of the internal lumen are exposed from the patterning mold and remaining surface portions of the internal lumen are covered by and in contact with the patterning mold;
forming a first electrode layer over the first and second surface portions exposed from the patterning mold using electroless deposition, the first electrode layer comprising a first metal; and
after the forming, removing the patterning mold from the internal lumen.

2. The method of claim 1, wherein:
the first electrode layer is a seed layer of the first metal; and
the method further comprises, after the forming, electroplating a second metal on the seed layer to form a first electrode over the first surface portion and a second electrode over the second surface portion.

3. The method of claim 2, wherein the first metal comprises nickel and the second metal comprises gold.

4. The method of claim 2, wherein after the electroplating, the first and second electrodes extend from a first region of the internal lumen proximal to the first longitudinal end to a second region of the internal lumen proximal to the second longitudinal end.

5. The method of claim 1, further comprising, after the forming, forming a protective layer over the first electrode layer.

6. The method of claim 5, wherein the protective layer comprises parylene C and has a cross-sectional thickness of 1 μm or less.

7. The method of claim 1, further comprising, prior to the forming, coating the internal lumen with an adhesion promoting layer to form the first, second, and remaining surface portions of the internal lumen.

8. The method of claim 7, wherein the adhesion promoting layer comprises parylene C.

9. The method of claim 1, further comprising, prior to the forming, subjecting the internal lumen to an adhesion promotion treatment.

10. The method of claim 9, wherein the adhesion promotion treatment comprises exposure to oxygen plasma, exposure to ultraviolet-ozone, coating with a coupling agent, or any combination of the foregoing.

11. The method of claim 1, wherein the removing comprises:
modifying a shape of the patterning mold such that the first, second, and remaining surface portions are exposed; and
displacing the patterning mold longitudinally toward the first longitudinal end of the catheter.

12. The method of claim 1, wherein the removing comprises melting, dissolving, or etching the patterning mold while retained within the internal lumen.

13. The method of claim 1, wherein after the forming, the first metal on the first and second surface portions forms an interdigitated electrode pattern.

14. The method of claim 1, wherein the first metal comprises nickel, and the electroless deposition comprises:
(a) exposing the internal lumen to a first solution of stannous chloride and trichloroacetic acid so as to bond tin ions thereto;
(b) after (a), exposing the internal lumen to sodium tetrachloropalladate solution to replace the tin ions with palladium; and
(c) after (b), exposing the internal lumen to an electroless nickel plating solution to form the first electrode layer using the palladium as nucleation sites for the nickel wherein (a) is prior to or after the inserting.

15. A system comprising:
a catheter constructed to be disposed within an in vivo environment, the catheter having a first longitudinal end, a second longitudinal, and an internal lumen extending between the first and second longitudinal ends; and
first and second electrodes integrally formed over respective surface portions of the internal lumen, each of the first and second electrodes comprising an electroless-deposited layer of a first metal.

16. The system of claim 15, wherein each of the first and second electrodes further comprises an electroplated layer of a second metal.

17. The system of claim 15, further comprising:
an adhesion promoting layer disposed between the catheter and the first and second electrodes along a radial direction of the catheter;
a protective layer formed over the first and second electrodes; or
both of the above.

18. The system of claim 15, further comprising:
a voltage source configured to apply AC voltage signals to the first and second electrodes; and
a controller comprising one or more processors and a computer readable storage media storing instructions that, when executed by the one or more processors, cause the one or more processors to:
control the voltage source to apply an AC voltage signal to the first and second electrodes and receive a measurement signal indicative of an impedance value measured between the first and second electrodes; and
determine a state of bacteria growth on the catheter based at least in part on the measurement signal.

19. The system of claim 15, wherein the first and second electrodes form an interdigitated electrode pattern.

20. The system of claim 15, wherein the catheter is a urinary catheter formed of silicone rubber, latex, polytetrafluoroethylene (PTFE), polyvinyl chloride (PVC), or any combination of the foregoing.
